(19) United States
(12) Patent Application Publication
(10) Pub. No.: US 2026/0041627 A1
(43) Pub. Date: Feb. 12, 2026

(54) TREATMENTS FOR EYE CONDITIONS AND DRYNESS

(71) Applicant: PS Therapy, Inc., Westmont, IL (US)

(72) Inventor: Siddharth Narendran, Tamil Nadu (IN)

(21) Appl. No.: 19/292,539

(22) Filed: Aug. 6, 2025

(30) Foreign Application Priority Data
Aug. 7, 2024 (IN) 202441059596

(51) Int. Cl. A61K 9/00; A61P 27/12

(52) U.S. Cl. CPC A61K 9/00; A61P 27/12

(57) ABSTRACT

The present invention is directed to methods for treating dry eye comprising administering indole-3-carbinol, a metabolite thereof, a derivative thereof, a salt thereof or a hydrate thereof to a subject in need thereof. The present invention is further directed to treating vulvovaginal dryness comprising administering indole-3-carbinol, a metabolite thereof, a derivative thereof, a salt thereof or a hydrate thereof to a subject in need thereof. The present invention is further directed to treating cataracts and/or glaucoma comprising administering an ophthalmic composition comprising indole-3-carbinol, a metabolite, a derivative, a salt or a hydrate thereof to a subject in need thereof, wherein the cataracts and/or glaucoma are associated with menopause.

### FIELD OF THE INVENTION

The present invention is directed to methods for treating dry eye comprising administering indole-3-carbinol, a metabolite thereof, a derivative thereof, a salt thereof or a hydrate thereof to a subject in need thereof. The present invention is further directed to treating vulvovaginal dryness comprising administering indole-3-carbinol, a metabolite thereof, a derivative thereof, a salt thereof or a hydrate thereof to a subject in need thereof. The present invention is further directed to treating cataracts and/or glaucoma comprising administering an ophthalmic composition comprising indole-3-carbinol, a metabolite, a derivative, a salt or a hydrate thereof to a subject in need thereof, wherein the cataracts and/or glaucoma are associated with menopause.

### BACKGROUND OF THE INVENTION

Eye conditions including dry eye disease and retinal disorders afflict 100's of millions of people worldwide. Dry eye disease is an ocular disease affecting approximately 10-20% of the population. Over 350 million people worldwide suffer from visual impairment or blindness due to retinal diseases and disorders. These numbers are only expected to rise due to aging populations in many countries. Despite the huge population of afflicted individuals there are a dearth of treatments or preventative medicines available to combat dry eye disease and retinal diseases and disorders.

As mentioned above, dry eye disease likely afflicts over a billion people worldwide. In addition, various subsets of this population, including contact lens wearers and patients undergoing glaucoma treatment for example, have a high incidence of dry eye. In individuals suffering from dry eye, the protective layer of tears that normally protects the ocular surface is compromised. This compromise is a result of insufficient or unhealthy production of one or more tear components. These insufficiencies are typically categorized as reduced aqueous synthesis, known as aqueous deficiency, or lipid layer deficient, known as meibomian gland deficiency (“MGD”). These deficiencies can lead to compromised tear layer exposure of the surface of the eye, ultimately promoting desiccation and damage of surface cells.

Signs and symptoms of dry eye disease include but are not limited to conjunctival and corneal staining, redness, blurry vision, decreased tear film break-up time, decreased tear production, tear volume, and tear flow, abnormal tear layer composition, increased conjunctival redness, excess debris in the tear film, ocular dryness, ocular grittiness, ocular burning, foreign body sensation in the eye, excess tearing, photophobia, ocular stinging, refractive impairment, ocular sensitivity, and ocular irritation. Patients may experience one or more of these symptoms. The excess tearing response may seem counterintuitive to dry eye, however excess tearing is a natural response to the irritation and foreign body sensation caused by dry eye. Some people may also experience ocular itching due to a combination of ocular allergies and dry eye.

There are many possible variables that can influence a person's signs or symptoms of dry eye including levels of circulating hormones, various autoimmune diseases (e.g. Sjögren's syndrome and systemic lupus erythematosus), cutting of sensory corneal nerve plexus reducing corneal sensation such as from ocular surgeries including PRK or LASIK®, many medications, environmental conditions, visual tasking with reduced blinking such as computer use, ocular fatigue, and mechanical influences such as reduced corneal sensitivity, partial lid closure during sleep, surface irregularities (e.g. pterygium), and other lid or ocular surface irregularities (e.g. ptosis, entropion/ectropion, pinguecula).

There are a number of products commercially available for the treatment of dry eye. However, such products provide only temporary relief of acute symptoms. These products typically last for a few minutes and in rare cases up to ten minutes. Further, these product provide virtually none of the known inotropic excipients present in tears that promote the healthiest ocular surface, such as growth factors, lipid solubilizers, antimicrobials and other anti-inflammatory components of a healthy normal tear film. Artificial tears are suitable for short term use only and provide virtually no long-term health benefit. In addition, these products may themselves cause ocular discomfort upon installation in the eye and or with prolonged or chronic repeat use. For example, artificial tears and ointments may provide temporary relief of dry eye but do little to arrest or reverse any damaging conditions.

For more severe cases of dry eye, in which the cornea is inflamed, anti-inflammatory agents are sometimes prescribed. Topical corticosteroids (in eye drops) are safe for short-term use to combat inflammation, but can cause side effects, including but not limited to decreased wound healing, cataract, and in some cases, increased risk of elevated intra-ocular pressure in patients. Likewise, nonsteroidal anti-inflammatory drugs (NSAIDs) in their current ophthalmic dosage forms are approved for short term use only. For example, the use of NSAIDs to treat inflammation and pain associated with post ocular-surgery may result in corneal damage in patients predisposed to such conditions. Further the use of NSAIDs may delay wound healing after repeated dosing, or ocular discomfort.

Commercial cyclosporin-A (Restasis®-Allergan) is the first approved therapeutic agent for the treatment of dry eye and is suitable for long term use. However, the primary side effect cited on the package insert is ocular burning and stinging upon instillation, and Restasis® was shown to be effective in only 17% of patients. To improve patient discomfort during the induction phase of cyclosporin therapy, clinicians may prescribe topical corticosteroids or NSAIDs (in eye drop form) in conjunction with cyclosporin-A. However, it takes about six months for just 15% of dry eye patients treated to demonstrate a 10 mm improvement in their tearing as measured by experts in the art versus vehicle. Autologous serum, on the other hand, in which blood is drawn from a patient and cells are spun off harvesting the plasma which is then frozen until use, contains within most if not all of the beneficial inotropic factors found in tears. Autologous serum has been the most effective treatment to date for severe dry eye such as that associated with Sjogren's syndrome.

Causes of dry eye are multiple and include environmental exposure to excessively dry air, airborne particulates and pollutants, smoke, smog, ultra-violet radiation, environmental pollutants, keratoconjunctivitis sicca, Sjorgen's syndrome, corneal injury, age-related dry eye, Stevens-Johnson syndrome, congenital alachrima, pharmacological side effects, infection, Riley-Day syndrome, conjunctival fibrosis, eye stress, glandular and tissue destruction, ocular cicatrical pemphogoid, blepharitis, autoimmune and other immunodeficient disorders, allergies, diabetes, lacrimal gland deficiency, lupus, Parkinson's disease, Sjogren's syndrome, rheumatoid arthritis, rosacea, environmental exposure to excessively dry air, airborne particulates, smoke, and smog and inability to blink.

Menopausal women, including perimenopausal and postmenopausal women can experience several eye-related conditions such as cataracts, glaucoma and dry eye due to hormonal changes. The decrease in estrogen levels during menopause has been linked to a higher prevalence of these eye conditions.

Genitourinary symptoms are common in postmenopausal women as well, including vaginal dryness. Ettinger B, et al., Measuring symptom relief in studies of vaginal and vulvar atrophy: the most bothersome symptom approach. Menopause, 2008 September-October, 15(5), 885-889. It is estimated that 84.2% of woman at 6 years post menopause will manifest genitourinary syndrome of menopause (“GSM”), with 100% of those reporting vaginal dryness. Palma F, et al., Vaginal atrophy of women in postmenopause. Results from a multicentric observational study: The AGATA study. Maturitas, 2016 January, 83, 40-44.

Treatments of vaginal dryness have focused on increasing the thickness of the tissues and/or lowering the vaginal pH. Treatments effective in achieving these objectives have included estrogens (topically or systemically) or the selective estrogen receptor modulator, ospemifene. Sturdee D W, et al., Recommendations for the management of postmenopausal vaginal atrophy, Climacteric 2010 December, 13(6), 509-522; Bachmann G A, et al., Ospemifene Study Group. Ospemifene effectively treats vulvovaginal atrophy in postmenopausal women: results from a pivotal phase 3 study, Menopause, 2010 May-June, 17(3), 480-486. Non-hormonal therapies include vaginal lubricants and moisturizers, which do not reverse atrophic changes but enhance comfort. To date, there is no safe, effective form of treating vaginal dryness.

Indole-3-carbinol has been studied as a possible cancer treatment. However, indole-3-carbinol has not been studied to treat dry eye or retinal diseases or disorders.

Thus, there is a need in the art for additional therapeutic treatments for ophthalmic diseases, disorders and conditions including dry eye and retinal diseases and disorders. Further, there is a need in the art for treating vaginal dryness.

### SUMMARY OF THE INVENTION

The present invention is directed to methods for treating dry eye comprising administering indole-3-carbinol, a metabolite thereof, a derivative thereof, a salt thereof or a hydrate thereof to a subject in need thereof.

The present invention is further directed to methods for treating vulvovaginal dryness comprising administering indole-3-carbinol, a metabolite thereof, a derivative thereof, a salt thereof or a hydrate thereof to a subject in need thereof.

The present invention is further directed to methods for treating cataracts comprising administering an ophthalmic composition comprising indole-3-carbinol, a metabolite, a derivative, a salt or a hydrate thereof to a subject in need thereof, wherein the cataracts are associated with menopause.

The present invention is further directed to methods for treating glaucoma comprising administering an ophthalmic composition comprising indole-3-carbinol, a metabolite, a derivative, a salt or a hydrate thereof to a subject in need thereof, wherein the glaucoma is associated with menopause.

### BRIEF DESCRIPTION OF THE DRAWINGS

FIG. 1a-d. Slit lamp analysis of fluorescein-stained corneal epithelium showing untreated (1a) and 1(c) mouse eyes and mouse eyes treated with 100 micromolar of indole-3-carbinol (1b) and 1 millimolar of indole-3-carbinol 1(d), wherein the mouse eyes were previously treated with scopolamine.

FIG. 2. Bar chart demonstrating the fold change from control of ICAM-1 mRNA expression levels wherein I3C denotes indole-3-carbinol, LE denotes the untreated left mouse eye and RE denotes the treated right mouse eye.

FIG. 3. Bar chart demonstrating the fold change from control of IL-6 mRNA expression wherein I3C denotes indole-3-carbinol

FIG. 4. Bar chart demonstrating the fold change from control CYP1A1 mRNA expression wherein I3C denotes indole-3-carbinol.

FIG. 5. Bar chart demonstrating the fold change from control IL-6 mRNA expression wherein I3C denotes indole-3-carbinol.

FIG. 6. Bar chart demonstrating the fold change from control of IL-1β mRNA expression wherein I3C denotes indole-3-carbinol.

FIG. 7. Bar chart demonstrating the fold change from control CYP1A1 mRNA expression wherein I3C denotes indole-3-carbinol.

### DETAILED DESCRIPTION OF THE INVENTION

The Applicant has surprisingly discovered that indole-3-carbinol treats dry eye. The Applicant has further surprisingly discovered that indole-3-carbinol will likely treat vulvovaginal dryness. The Applicant has further surprisingly discovered that indole-3-carbinol modifies estrogen levels such that cataracts and glaucoma associated with menopause are treated.

In one embodiment, the present invention is directed to methods for treating dry eye comprising administering indole-3-carbinol, a metabolite thereof, a derivative thereof, a salt thereof or a hydrate thereof to a subject in need thereof.

In a preferred embodiment, the present invention is directed to methods for treating dry eye comprising topically administering indole-3-carbinol, a metabolite thereof, a derivative thereof, a salt thereof or a hydrate thereof to a subject in need thereof.

In a more preferred embodiment, the present invention is directed to methods for treating dry eye comprising topically administering indole-3-carbinol, a metabolite thereof, a derivative thereof, a salt thereof or a hydrate thereof to an eye of a subject in need thereof.

In another embodiment, the present invention is directed to methods for treating dry eye, comprising topically administering indole-3-carbinol, a metabolite thereof, a derivative thereof, a salt thereof or a hydrate thereof to an eye of a subject in need thereof, wherein dry eye is secondary to ocular surface inflammation.

In a preferred embodiment, topically administering indole-3-carbinol, a metabolite thereof, a derivative thereof, a salt thereof or a hydrate thereof to an eye of a subject in need thereof results in a greater reduction in ocular surface inflammation than administration of cyclosporine.

In another preferred embodiment, topically administering indole-3-carbinol, a metabolite thereof, a derivative thereof, a salt thereof or a hydrate thereof to an eye of a subject in need thereof results in a greater tear production than administration of cyclosporine.

In another embodiment, the present invention is directed to methods for treating dry eye caused by menopause, keratoconjunctivitis sicca, Sjorgen's syndrome, corneal injury, age-related dry eye, Stevens-Johnson syndrome, congenital alachrima, pharmacological side effects, infection, Riley-Day syndrome, conjunctival fibrosis, eye stress, glandular and tissue destruction, ocular cicatrical pemphogoid, blepharitis, autoimmune and other immunodeficient disorders, allergies, diabetes, lacrimal gland deficiency, lupus, Parkinson's disease, Sjogren's syndrome, rheumatoid arthritis, rosacea, environmental exposure to excessively dry air, ultra-violet radiation exposure, airborne particulates, environmental pollutants, smoke, smog, inability to blink and combinations thereof.

In another embodiment, the present invention is directed to methods for treating vulvovaginal dryness comprising topically administering indole-3-carbinol, a metabolite thereof, a derivative thereof, a salt thereof or a hydrate thereof to an eye of a subject in need thereof.

In a preferred embodiment, the vulvovaginal dryness is associated with genitourinary syndrome of menopause comprising topically administering indole-3-carbinol, a metabolite thereof, a derivative thereof, a salt thereof or a hydrate thereof to an eye of a subject in need thereof.

Administration of indole-3-carbinol may occur topically, orally or vaginally. In a preferred embodiment, indole-3-carbinol is applied topically to the surface of the eye.

Indole-3-carbinol (CAS #458-37-7) has the following chemical structure

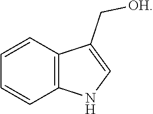 [SMILES: OCc1c[nH]c2ccccc12]

Metabolites of indole-3-carbinol include, but are not limited to, 3,3′-diindolylmethane.

3,3′-diindolylmethane (CAS #1968-05-4) has the following chemical structure

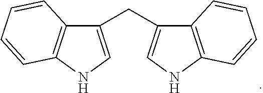 [SMILES: c1ccc2c(Cc3c[nH]c4ccccc34)c[nH]c2c1]

Derivatives of indole-3-carbinol include, but are not limited to, indole[3,2-b]carbazole (ICZ) and indole-3-acetonitrile.

The compounds of the present invention can be used in the form of pharmaceutically acceptable salts derived from inorganic or organic acids or bases. The phrase “pharmaceutically acceptable salt” means those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, S. M. Berge et al. describe pharmaceutically acceptable salts in detail in J. Pharmaceutical Sciences, 1977, 66: 1 et seq.

The salts can be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, hyaluronic acid, malic acid, sulphuric acid and phosphoric acid and such organic acids as oxalic acid, malic acid, maleic acid, methanosulfonic acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylammonium, dimethylammonium, trimethylammonium, triethylammonium, diethylammonium, and ethylammonium among others. Other representative organic amines useful for the formation of base addition salts include ethylenediamine, ethanolamine, diethanolamine, piperidine, piperazine and the like.

In a preferred embodiment, indole-3-carbinol is applied to a subject in need thereof in an ophthalmic composition.

As used herein the term “treatment” or “treating” refers to preventing, slowing or reversing the progression of a disease, condition or disorder or reducing, alleviating or ameliorating symptoms of a disease, condition or disorder.

As used herein “vulvovaginal dryness” refers to dryness localized in the female reproductive tract, e.g., the vulva including the vulvar vestibule, vagina, and combinations thereof. The pain can be due to a medical condition, psychological condition and/or sexual activity.

As used herein the term “vulvovaginal” or “vulvovaginal region” refers to the vulva including the vulvar vestibule, the vagina, and combinations thereof.

As used herein “genitourinary syndrome of menopause” refers to a collection of symptoms and signs associated with a decrease in estrogen and other sex steroids involving changes to the labia majora/minora, clitoris, vestibule/introitus, vagina, urethra and bladder. The syndrome may include but is not limited to genital symptoms of dryness, burning, and irritation.

As used herein the term “effective amount” refers to the amount necessary to treat a patient in need thereof.

Throughout the application, the singular forms “a,” “an,” and “the” include plural reference unless the context clearly dictates otherwise.

Throughout the application, all disclosed ranges include all possible values within those ranges. All possible values within the ranges disclosed in the application can also be used as endpoints for additional ranges between these values.

The disclosed embodiments are simply exemplary embodiments of the inventive concepts disclosed herein and should not be considered as limiting unless the claims expressly state otherwise.

The following example is intended to illustrate the present invention and to teach one of ordinary skill in the art how to use the methods of the invention. The example is not intended to be limiting in any way.

### EXAMPLE

### Example 1—Treatment of Dry Eye Disease

### Method

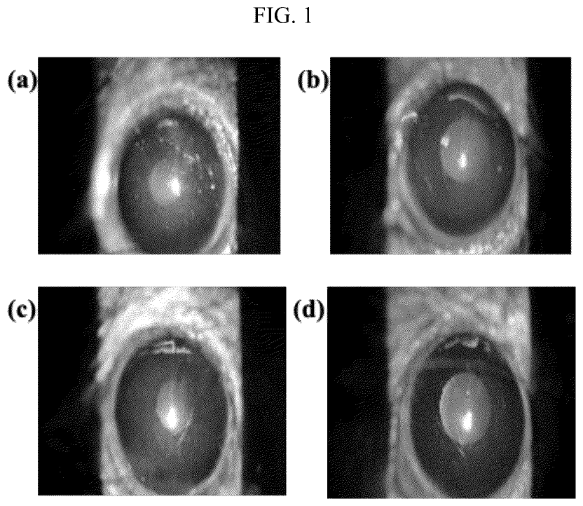

Dry eye disease was induced in mice by administering subcutaneous injections of 0.1 milliliters of scopolamine at 0.5 milligrams per milliliter three times per day for 14 days. Concurrently, indole-3-carbinol was instilled in the right eye of the mice at either 100 micromolar or 1 millimolar. As a control saline was instilled in the left eye of the mice. Following instillation, a baseline Schirmer's test (tear volume) was performed. On the 14^{th} day, corneal epithelial disruption was assessed using slit lamp analysis following 1% fluorescein staining. Subsequently, the mice were euthanized via ketamine overdose, and their eyes were enucleated, with the corneas isolated under sterile conditions. See FIG. 1 for results.

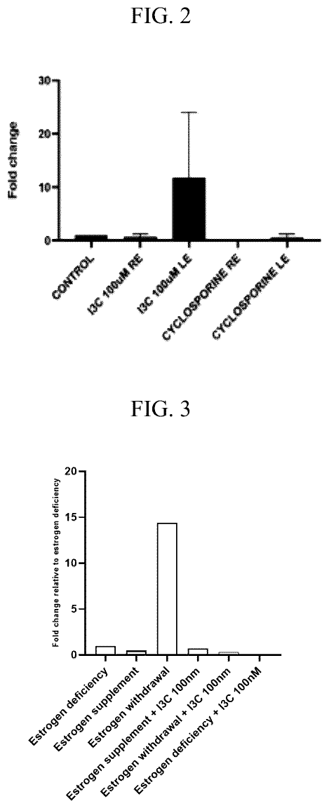

ICAM-1 plays a pivotal role in instigating ocular surface inflammation during hyperosmolar stress. The involvement of ICAM-1 in exacerbating the inflammatory cascade, leading to the subsequent release of cytokines, underscores its significance in dry eye disease. Thus, elevated ICAM-1 levels indicates induction of dry eye disease. Whereas reduction of ICAM-1 levels from the induced state demonstrates treatment of dry eye disease. Accordingly, mRNA expression for intercellular adhesion molecule 1 (“ICAM-1”), pro-inflammatory marker, was also assessed for the mice treated with 100 micromolar of indole-3-carbinol. As a positive control a set of mice were also treated with cyclosporine. See FIG. 2 and Table 1, below, for results.

TABLE 1

| Treatment | Fold Change from Control |
|---|---|
| Control | 1.00 |
| 100 μM indole-3-carbinol right eye | 0.74 |
| 100 μM indole-3-carbinol left eye | 11.78 |
| Cyclosporine right eye | 0.19 |
| Cyclosporine left eye | 0.62 |

### Results

As demonstrated in FIG. 1, mouse eyes treated with either 100 micromolar or 1 millimolar of indole-3-carbinol demonstrated less disruption of the corneal epithelium than mouse eyes treated with saline. Compare 1b with 1a and 1d with 1c.

As depicted in FIG. 2, levels of ICAM-1 are elevated in eyes induced with the disease, but are relatively reduced in eyes treated with indole-3-carbinol at 100 micromolar. Thus, indole-3-carbinol is effective at treating dry eye disease.

### Example 2—Treatment of Dry Eyes Associated with Menopause

### Method

Corneal and lens epithelial cells exhibit several changes due to hormonal fluctuations. To study the effects of estrogen on the cornea and the lens, corneal epithelial cells and human lens epithelial cells were cultured in phenol red free Dulbecco's modified Eagle medium along with charcoal treated fetal bovine serum (“FBS”) to perform hormonal manipulation. The cells were either cultured in phenol red free media and charcoal treated FBS (estrogen deficiency) or treated with 10 nanomolar 170-estradiol for 3 days. Next, cells were divided into the following groups: i) continue in phenol red free media and charcoal treated FBS supplemented with Estradiol; ii) withdrawal of Estradiol and cultivation in phenol red free media (i.e. estrogen withdrawal conditions (“EWD”)) iii) continue in phenol red free media and charcoal treated FBS supplemented with Estradiol with 100 nanomolar indole-3-carbinol; iv) withdrawal of Estradiol and cultivation in phenol red free media (EWD) with indole-3-carbinol; v) treatment with 100 nanomolar indole-3-carbinol in estrogen deficient culture; or vi) estrogen deficient group which was then continued for 2 days. At the end of the experiment, cells were lysed for RNA.

Interleukin-6 (“IL-6”) and interleukin-1β (“IL-1β”) plays a significant role in the pathogenesis of dry eye disease (“DED”) and are found elevated in the tears of patients with DED. Post-menopausal women exhibit higher IL-6 levels compared to pre-menopausal women, partly due to the decline in estrogen levels. Emerging evidence suggests that menopause-induced changes in pro inflammatory cytokines like IL-6 and IL-1β activity are factors that could significantly influence the ocular homeostasis.

Aryl hydrocarbon receptors (“AHR”) are involved in xenobiotic metabolism and also exert anti-inflammatory effects through its immune-regulatory potential. AHR is expressed in many ocular tissues such as retina, choroid, cornea and the orbit. The AHR genes like cytochrome P450 1A1 (CYP1A1), cytochrome P450 1A2 (CYP1A2) and cytochrome P450 1B1 (CYP1B1) are involved in detoxification. Therefore, their activation is crucial to maintain ocular homeostasis.

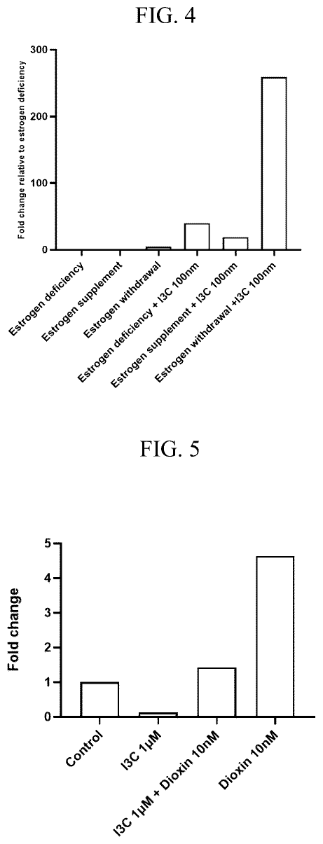

IL-6 and CYP1A1 levels were determined via RT-PCR and are presented in FIGS. 3 and 4, respectively. Expression levels are presented as fold change from GAPDH expression.

### Results

As demonstrated in FIG. 3, IL-6 expression increased in estrogen withdrawal group, and the treatment of the same with indole-3-carbinol at 100 nanomolar significantly reduced the expression of IL-6. Similarly, in FIG. 4, CYP1A1 expression decreased in estrogen withdrawal group, an AHR detoxifying gene. Treatment with indole-3-carbinol at 100 nanomolar increased its expression, indicating a positive expression of AHR. Thus, indole-3-carbinol can treat dry eye caused by menopause.

### Example 3—Treatment of Dry Eyes Arising Due to Pollution

### Method

The ocular surface is directly exposed to environmental pollutants, like 2,3,7,8-tetrachloro-dibenzo-p-dioxin (TCDD), referred to as dioxin, which is a persistent organic pollutant. It is a strong activator of AHR, leading to its persistent activation and inducing toxicological effects. Shorter-lived agonists like indole-3-carbinol activates the receptor momentarily and gets metabolized in negative feedback loop thereby contributing to anti-inflammatory activity.

Human corneal epithelial cells were cultured in DMEM and FBS. The cells were pre-treated with indole-3-carbinol at 1 micromolar for 2 days. Next the cells were treated with 10 nanomolar dioxin for 2 days. Cells were isolated and lysed for RNA.

IL-6, IL-1β and cytochrome P450 1A1 levels determined via RT-PCR and are presented in FIGS. 5, 6 and 7, respectively. Expression levels are presented as fold change from GAPDH expression.

### Results

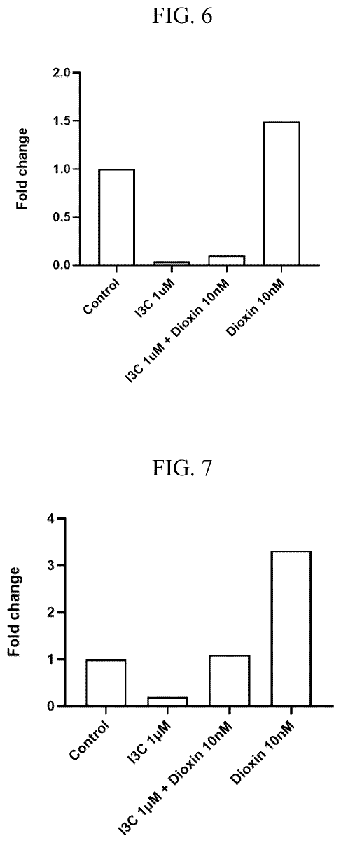

IL-6 and IL-1β expression levels as shown in FIGS. 5 and 6 are elevated in dioxin treatment group, which is reduced when dioxin exposed cells are treated with 1 micromolar indole-3-carbinol. This result demonstrates a reduction in pro-inflammatory cytokines.

Dioxin is a highly potent exogenous ligand of AHR and produces sustained AHR-CYP1A1 axis activation and is resistant to AHR-induced metabolism and breakdown. This activation leads to significant increase in CYP1A1 expression and activity, often associated with toxicological outcomes (Dioxin toxicity). Indole-3-carbinol is an AHR agonist and activates AHR-CYP1A1 axis, after which the indole-3-carbinol is metabolized. This transiently active state thereby lacks dioxin toxicity. As seen in FIG. 7, indole-3-carbinol reduces dioxin and thus treats dry eye due to environmental pollution.

What is claimed is:

1. A method of treating dry eye comprising administering indole-3-carbinol, a metabolite, a derivative, a salt or a hydrate thereof to a subject in need thereof.

2. The method of claim 1, wherein the indole-3-carbinol is administered topically.

3. The method of claim 2, wherein the indole-3-carbinol is applied to an eye of the subject in need thereof.

4. The method of claim 1 wherein dry eye is a secondary symptom to a disorder selected from the group consisting of menopause, ocular surface inflammation, keratoconjunctivitis sicca, Sjorgen's syndrome, corneal injury, age-related dry eye, Stevens-Johnson syndrome, congenital alachrima, pharmacological side effects, infection, Riley-Day syndrome, conjunctival fibrosis, eye stress, glandular and tissue destruction, ocular cicatrical pemphogoid, blepharitis, autoimmune and other immunodeficient disorders, allergies, diabetes, lacrimal gland deficiency, lupus, Parkinson's disease, Sjogren's syndrome, rheumatoid arthritis, rosacea, environmental exposure to excessively dry air, airborne particulates, environmental pollution, smoke, and smog, inability to blink and combinations thereof.

5. The method of claim 1, wherein administration results in a greater reduction in ocular surface inflammation than administration of cyclosporine.

6. The method of claim 1, wherein administration results in a greater tear production than administration of cyclosporine.

7. A method of treating dry eye comprising administering an ophthalmic composition comprising indole-3-carbinol, a metabolite, a derivative, a salt or a hydrate thereof to a subject in need thereof.

8. A method of treating vulvovaginal dryness comprising administering indole-3-carbinol, a metabolite, a derivative, a salt or a hydrate thereof to a subject in need thereof.

9. The method of claim 8, wherein the dyspareunia is associated with genitourinary syndrome of menopause.

10. A method of treating cataracts comprising administering an ophthalmic composition comprising indole-3-carbinol, a metabolite, a derivative, a salt or a hydrate thereof to a subject in need thereof, wherein the cataracts are associated with menopause.

11. A method of treating glaucoma comprising administering an ophthalmic composition comprising indole-3-carbinol, a metabolite, a derivative, a salt or a hydrate thereof to a subject in need thereof, wherein the glaucoma is associated with menopause.
